(19) United States
(12) Patent Application Publication
(10) Pub. No.: US 2006/0263240 A1
(43) Pub. Date: Nov. 23, 2006

(54) Electrolyzed water treatment for face and hands

(75) Inventor: John Hopkins, Orem, UT (US)

Correspondence Address: BRACEWELL & GIULIANI LLP, P.O. BOX 61389, HOUSTON, TX, 77208-1389, US

(73) Assignee: Electric Aquagenics Unlimited, Lindon, UT (US)

(21) Appl. No.: 11/429,768

(22) Filed: May 8, 2006

Related U.S. Application Data
(60) Provisional application No. 60/678,603, filed on May 6, 2005.

(51) Int. Cl. A61L 2/18

(52) U.S. Cl. 422/28; 134/42

(57) ABSTRACT

Embodiments of the present invention provide for a method of cleaning and disinfecting the skin using electrolyzed water. A particularly preferred embodiment provides for the application of Type C water to the skin. Additional preferred embodiments provide for the application of Type B water to the skin followed by the application of Type A water or Type C water to the skin.

### CROSS-REFERENCE TO RELATED APPLICATION

This non-provisional patent application claims the benefit under §119(e) of U.S. Provisional Patent Application Ser. No. 60/678,603, filed May 6, 2005, which is incorporated herein by reference.

### BACKGROUND OF THE INVENTION

1. Field of the Invention

This invention relates in general to cleaning and disinfecting. More particularly, this invention relates to methods for cleaning and disinfecting the face, hands, and skin using electrolyzed water.

2. Background of the Invention

Skin disinfectants are routinely used in professional and non-professional settings to kill microbes. A physician has a need to disinfect his or her skin both before and after examining a patient to avoid contracting or passing infections. Furthermore, prior to the performance of many medical procedures, a patient's skin must be cleaned and disinfected properly to avoid post-procedure infections. In non-professional settings, for example, a commuter riding public transportation may wish to disinfect her hands before handling food; a child playing in a park may need to clean his hands but not have the convenience of soap and water nearby; or an individual shopping in a grocery store may wish to clean his or her hands prior to handling food or after touching a shopping cart. Each of these situations requires, optimally, a skin disinfectant that is effective, easy to use, and safe.

Current commercial skin cleaners and disinfectants exhibit varying degrees of toxicity and can cause skin irritation. A number of skin disinfectants have been developed that contain alcohol as the primary antimicrobial agent. There are two general problems associated with alcohol-based disinfectants. First, the effective concentration of alcohol, generally greater than 60 percent (weight/volume percentage) ethanol or its equivalent, irritates the skin, causes dryness, and often leaves skin peeled and cracked. Second, repeated use of alcohol disinfectants can increase susceptibility to infection because dried, chapped skin tends to be more susceptible to microbial contamination. Furthermore, whereas alcohol can be an effective disinfectant, once it evaporates its antimicrobial activity is lost.

Hand disinfectants that contain short-chain aliphatic alcohols are often used to prevent cross-infection in health care environments. The irritant potential of these disinfectants is not well known. Skin tolerance is a major compliance factor and a high proportion of health care workers suffer from low-grade irritant contact dermatitis. The most important risk factor for occupational contact dermatitis in hospital personnel is exposure to irritants such as detergents and alcohol-based solutions. These prior art skin cleaners and disinfectants evidence an unmet need for an alternative skin cleaner and disinfectant that is nontoxic, nonirritating, effective, and safe.

Electrolyzed water is useful for disinfecting and cleaning. Electrolyzed water is produced by electrolysis. A feed water solution containing a saline solution component is supplied to an electrolytic cell comprising both an anode chamber and a cathode chamber. When normal culinary tap water that has been treated is combined with an electrolyte (i.e., salt) and placed in contact with an electrical probe or plate, electrolysis occurs once the probe or plate is electrically charged by a power source. The probes or plates are separated by a membrane that isolates and separates certain chemical ions. During the chemical reaction, positively charged ions naturally migrate to the negative electrode (i.e., cathode) and negatively charged ions including chloride (Cl⁻¹) naturally migrate towards the positive electrode (i.e., anode). The feed water solution is cathodically electrolyzed in the cathode chamber to produce electrolyzed water as an antioxidant solution called alkaline catholyte, commonly referred to as Type B water. The feed water solution is anodically electrolyzed in the anode chamber to produce electrolyzed water as an oxidant solution called anolyte, whose pH is modified in the process, and is commonly referred to as Type A water. The anolyte is a strong oxidizing solution. More specifically, acidic electrolyzed water is normally generated from the anode electrode through electrolysis of a dilute aqueous sodium chloride (NaCl) solution. Type A water has a very high ORP because of its high concentration of hydroxyl radicals, chlorine free radicals, and HOCl. During electrolysis of sodium chloride solutions, chloride (Cl⁻¹) and hydro-peroxides (H2O2) ions are electrochemically oxidized on the anode surface. These transition compounds further react to form HOCL and hydroxyl radicals. Hydroxyl radicals (OH) are the neutral form of the hydroxide ion. Hydroxyl radicals are highly reactive and have the highest oxidation-reduction potential of any known compound. They are an important part of radical and electrochemistry.

The relatively high bactericidal activity of acidic electrolyzed water, or Type A water, is attributed to high oxidation-reduction potential (ORP), HOCl, OCl, acidic pH, and the presence of dissolved Cl₂. The high ORP and low pH of Type A solution kills microbes on the cellular level. Every living cell is comprised of cytoplasmic materials. Embedded in the cytoplasmic materials of the cell wall are proteins that regulate the cell's functions, such as its temperature, nutritional inflow, and defenses, by receiving electric and chemical signals from the cell's organelles. The Type A high ORP, low pH solution interacts with the proteins. This interaction inhibits the organelles' signals to the proteins, thereby disrupting the protein's ability to open and shut the membrane portals. This leaves the cell's membrane portals stuck in an “open” position, which allows infiltration of the Type A low pH solution inside the cell walls, oxidizing the organelles and other biological matter in the cell. The HOCl in the Type A solution accelerates the organelles' oxidation. With the cell's membrane portals open the Type A high ORP, low pH solution can flood the cell and cause an osmotic or hydration overload within the cell. The Type A high ORP, low pH solution floods the cell faster than the cell can expel the fluid thus causing the cell to burst. Also contributing to the relatively high bactericidal activity is the presence of so-called “free available” chlorine, which comprises dissolved HOCl, Cl₂, and OCl⁻. The bactericidal activity of dissolved Cl₂ lessens over time as it evaporates or is otherwise lost from the Type A water during storage or a period of treatment. This loss may also affect other important properties of Type A water, such as its pH and ORP. Finally, the low pH of Type A water effectively, kills many pathogens.

### SUMMARY OF THE INVENTION

The present invention relates to methods for cleaning the face, hands, and skin using electrolyzed water. Skin cleaning and disinfecting with electrolyzed water overcomes many of the disadvantages of current skin cleaning and disinfecting methods. Electrolyzed water is more pathogenically effective, less irritating, safer, and lower in cost.

It has been found that electrolyzed water is highly efficacious, achieving higher kill rates of harmful pathogens than alternative cleaners and disinfectants. In tests conducted at a major university, electrolyzed water solutions achieved as high as a 6 log (99.9999%) reduction in Salmonella and E. coli on surfaces. Electrolyzed water is capable of killing bacteria, viruses, spores, and molds within seconds of contact. Furthermore, in contrast to other cleaners and disinfectants, pathogens are unlikely to become resistant to electrolyzed water over time.

Embodiments of the present invention provide for a method of cleaning and disinfecting the skin using electrolyzed water. A particularly preferred embodiment provides for the application of a stabilized form of Type A water to the skin. Additional preferred embodiment provide for the application of Type B water to the skin followed by the application of Type A water or a stabilized form of Type A water to the skin.

### DETAILED DESCRIPTION

Although the following detailed description contains many specific details for purposes of illustration, any person of ordinary skill in the art will appreciate that many variations and alterations to the following details are within the scope of the invention. Accordingly, the exemplary embodiment of the invention described below is set forth without any loss of generality to, and without imposing limitations thereon, the claimed invention.

Types of Electrolyzed Water

Electrolyzed water produced by electrolysis is classified into three types: Type A, Type B, and Type C. In the preferred embodiments described below, electrolyzed water is produced from an electrolyte solution made by combining tap or other water to create a solution with a concentration of about 0.05% to 0.3% sodium chloride (NaCl) by weight. Electrolyte solutions for producing electrolyzed water also may include potassium chloride (KCl), magnesium chloride (MgCl₂), sodium phosphate (NaH₂PO₄), and amidosulfonic acid (H₃NO₃S).

Type A water is a disinfectant that kills a large variety of bacteria, viruses, molds, and spores within seconds of contact. It is capable of replacing chlorinated water, and can be more effective at killing pathogens without toxicity. When positively charged ions migrate to the cathode, the fluid around the cathode develops a reduced pH in the approximate range of 1.8 to 3.4 and an ORP in the approximate range of 1000 to 1400⁺ millivolts (mV). Type A water can be produced as a continuous stream of clear solution having a pH of 1.8-3.4, an ORP of 1,000-1,400⁺ mV, and containing 8-70 parts per million (ppm) of HOCl. When Type A water comes in contact with organic material its pH increases, its ORP drops, and the HOCl and hydroxyl radicals oxidize the organic material, thus returning to ordinary water having a small amount of sodium chloride. Safety and toxicity tests have shown that Type A water is nontoxic at an HOCl concentration ranging from 10 to 70 ppm, a pH of 2.2-3.2, and an ORP ranging from 1135-1190⁺ mV.

Type B water is an extremely effective emulsifier and cleaner that has antimicrobial properties. It is capable of saponifying lipids upon contact. Type B water is an alkaline water stream and can be produced as a continuous stream of clear solution produced around the positive electrode, i.e., anode, during electrolysis. Type B water is basic with a pH in the approximate range of 10.5 to 12.0. The ORP of Type B water is in the approximate range of 600⁻-950⁻ mV. Type B water also contains sodium hydroxide (NaOH) ions in the approximate range of 8 to 50 ppm. NaOH has the ability to saponify, or create a microscopic “soap” film on the surface of a target. Type B water is effective in emulsifying oils and lipids and leaves no residue. Safety and toxicity tests show that Type B water is nontoxic at a pH of 10.5 to 12.0 and an ORP from 900⁻ to 950⁻ mV.

Type C water is essentially a form of stabilized Type A water with a longer shelf life. Type C water has an ORP in the approximate range of 850-1150⁺ mV, a pH value in the approximate range of 3.5-6.0, and contains HOCl in the approximate range of 8-70 ppm. Type C water is produced by recycling Type B water into the feed water solution used to make electrolyzed water. A particularly preferred method of producing Type C water is disclosed in United States Patent Application Publication No. 2006/0076248, which is incorporated herein by reference.

Table 1 summarizes the typical physical characteristics of Type A, B, and C water produced from an electrolyte solution containing sodium chloride.

TABLE 1
Physical Characteristics of Type A, B, and C Water

| Type | pH | ORP (mV) | HOCl (ppm) | NaOH (ppm) |
|---|---|---|---|---|
| Type A | 1.8-3.4 | 1000⁺-1400⁺ | 8-70 | — |
| Type B | 10.5-12.0 | 600⁻-950⁻ | — | 8-50 |
| Type C | 3.5-6.0 | 850⁺-1150⁺ | 8-70 | — |

Test Results

Electrolyzed water was found to be non-toxic and non-irritating.

Cytoxicity

In vitro biocompatibility tests were conducted using Type A water and Type B water to determine the potential for cytotoxicity. The Type A water used in the tests had a pH of 2.2-2.4, an ORP reading of 1130⁺ mV, and a beginning HOCl concentration of 8-10 ppm. The Type B water used in the tests had a pH of 10-11 and an ORP reading of 850⁻ mV. The solutions were applied at room temperature to filter disc samples. The test cell cultures were examined macroscopically for cell decolorization around the test article and controls, and to determine the zone of cell lysis. The cell monolayers also were examined microscopically to verify any decolorized zones and to determine cell morphology in proximity to the article. No evidence of cell lysis or toxicity was seen using either Type A water or Type B water. The testing was conducted by a third party testing organization and the control results for each test support the test results. The results are summarized in Tables 1-3. Those of skill in the art will recognize that the test results for Type A water are applicable to Type C water because Type C water is essentially a form of stabilized Type A water.

TABLE 1
Criteria for Cytotoxicity

| Grade | Reactivity | Condition of Cultures |
|---|---|---|
| 0 | None | No detectable zone around or under specimen |
| 1 | Slight | Some malformed or degenerated cells under |
| | | specimen |
| 2 | Mild | Zone limited to area under specimen and up to |
| | | 4 mm |
| 3 | Moderate | Zone extends 5-10 mm beyond specimen |
| 4 | Severe | Zone extends greater than 10 mm beyond |
| | | specimen |

TABLE 2
Type A Water

| | ZONE OF | | |
|---|---|---|---|
| ARTICLES | LYSIS (mm) | GRADE | REACTIVITY |
| Test Filter Disc (3 tests) | 0 | 0 | None |
| Filter Disc Control (3 tests) | 0 | 0 | None |
| Negative Control (3 tests) | 0 | 0 | None |
| Positive Control (3 tests) | 5 | 3 | Moderate |

TABLE 3
Type B Water

| | ZONE OF | | |
|---|---|---|---|
| ARTICLES | LYSIS (mm) | GRADE | REACTIVITY |
| Test Filter Disc (3 tests) | 0 | 0 | None |
| Filter Disc Control (3 tests) | 0 | 0 | None |
| Negative Control (3 tests) | 0 | 0 | None |
| Positive Control (3 tests) | 5 | 3 | Moderate |

Irritation and Sensitization

Rabbit studies were conduced using Type A water and Type B water to evaluate the potential for primary skin irritation and sensitization. The Type A water used in the tests had a pH of 2.2-2.4, an ORP reading of 1130⁺ mV, and a beginning HOCl concentration of 8-10 ppm. The Type B water used in the tests had a pH of 10-11 and an ORP reading of 850⁻ mV. The solutions were applied at room temperature to a skin sample on several rabbits. One day prior to treatment, the fur on each rabbit's back was clipped to the skin. Two 0.5 ml portions of the test article and control article were topically applied to the rabbits' skin, left in place for a period of 24 hours, and then removed. The rabbit is specified as an appropriate animal model for evaluating skin irritants by the current ANSI/AAMI/ISO testing standards. The skin sample sites were graded for erythema and edema at 1, 24, 48, and 72 hours after removal of the single sample application. The Primary Irritation Index was calculated following test completion for each rabbit. The erythema and edema scores obtained at the 24, 48, and 72 hour intervals were added together and divided by the total number of observations. This calculation was conducted separately for the test and control article for each rabbit. The score for the control was subtracted from the score for the test article to obtain the Primary Irritation Score. The Primary Irritation Score for each rabbit was added together and divided by the number of rabbits to obtain the Primary Irritation Index. Under the conditions of this study, for both Type A water and Type B water, the Primary Irritation Index resulting from the tests was calculated to be 0.0. No erythema and no edema were observed on the skin of any of the rabbits. No irritation was observed on the rabbits' skin. Testing was conducted by a third party testing organization and the control results for each test support the test results. The results are summarized in Tables 4-9. Those of skill in the art will recognize that the test results for Type A water are applicable to Type C water because Type C water is essentially a form of stabilized Type A water.

TABLE 4
Classification system for skin reaction

| | NUMERICAL |
|---|---|
| REACTION | GRADING |
| Erythema and Eschar Formation | |
| No erythema | 0 |
| Very slight erythema (barely perceptible) | 1 |
| Well-defined erythema | 2 |
| Moderate erythema | 3 |
| Severe erythema (beet redness) to eschar | 4 |
| formation preventing grading of erythema | |
| Edema Formation | |
| No edema | 0 |
| Very slight edema (barely perceptible) | 1 |
| Well-defined edema (edges of area well-defined by | 2 |
| definite raising) | |
| Moderate edema (raised approximately 1 mm) | 3 |
| Severe edema (raised more than 1 mm and extending | 4 |
| beyond exposure area) | |
| Total possible score for irritation | 8 |

TABLE 5
Irritation response categories

| RESPONSE | MEAN |
|---|---|
| CATEGORY | SCORE |
| Negligible | 0.0 to 0.4 |
| Slight | 0.5 to 1.9 |
| Moderate | 2.0 to 4.9 |
| Severe | 5.0 to 8.0 |

TABLE 6
Type A Water Dermal Observations

| | | | | Interval (hours) | | | | | | | |
|---|---|---|---|---|---|---|---|---|---|---|---|
| Rabbit | Weight | | | 1 | | 24 | | 48 | | 72 | |
| Number/Gender | (Kg) | Group | Observation | Left | Right | Left | Right | Left | Right | Left | Right |
| 66490 | 2.5 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |
| 66507 | 2.6 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |
| 66509 | 2.5 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |

TABLE 7
Type B Water Dermal Observations

| | | | | Interval (hours) | | | | | | | |
|---|---|---|---|---|---|---|---|---|---|---|---|
| Rabbit | Weight | | | 1 | | 24 | | 48 | | 72 | |
| Number/Gender | (Kg) | Group | Observation | Left | Right | Left | Right | Left | Right | Left | Right |
| 66490 | 2.5 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |
| 66507 | 2.6 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |
| 66509 | 2.5 | Test | Erythema | 0 | 0 | 0 | 0 | 0 | 0 | 0 | 0 |
| Male | | | Edema | 0 | 0 | 0 | 0 | 0 | 0 | 0 | 0 |
| | | Control | Erythema | 0 | 0 | 0 | 0 | 0 | 0 | 0 | 0 |
| | | | Edema | 0 | 0 | 0 | 0 | 0 | 0 | 0 | 0 |

TABLE 8
Type A Water Results

| | | | | Combined | Primary | |
|---|---|---|---|---|---|---|
| | | Control | | Primary | Irritation | |
| Rabbit | Test Score | Score | Individual Primary | Irritation | Index | Response |
| Number | Average | Average | Irritation Source | Source | (CPIS ÷ 3) | Category |
| 66490 | 0.0 | 0.0 | 0.0 | 0.0 | 0.0 | Negligible |
| 66507 | 0.0 | 0.0 | 0.0 | | | |
| 66509 | 0.0 | 0.0 | 0.0 | | | |

TABLE 9
Type B Water Results

| | | | | Combined | Primary | |
|---|---|---|---|---|---|---|
| | | Control | | Primary | Irritation | |
| Rabbit | Test Score | Score | Individual Primary | Irritation | Index | Response |
| Number | Average | Average | Irritation Source | Source | (CPIS ÷ 3) | Category |
| 66490 | 0.0 | 0.0 | 0.0 | 0.0 | 0.0 | Negligible |
| 66507 | 0.0 | 0.0 | 0.0 | | | |
| 66509 | 0.0 | 0.0 | 0.0 | | | |

### EXAMPLES

The examples that follow describe preferred methods for the application of electrolyzed water to the face, hands, and skin. Many variations on the specific perimeters of the examples are possible. Thus the examples are provided only for completeness, and not by way of limitation.

### Example 1

Type B water is applied to clean the face, hands, or skin followed by Type A water to disinfect the face, hands, or skin. A fluid wash using Type B water is applied having a pH of 10.5-12.0, an ORP reading of 600⁻ to 950⁻ mV, and a beginning sodium hydroxide concentration in the range of 8-50 ppm. In a preferred embodiment, the Type B water has a pH of 10-11 and an ORP reading of 850⁻ mV. The solution is preferably sprayed for several seconds. The spray wash using Type B water saponifies the lipids on the surface of the skin. Within seconds after saponification from applying the Type B water, the Type A water is applied to the skin to disinfect. The Type A water is applied having a pH of 1.8-3.4, an ORP reading of 1000⁺ mV to 1400⁺ mV, and a beginning HOCl concentration of 8-70 ppm. In a preferred embodiment, the Type A water has a pH of 2.2-2.4, an ORP reading of 1130⁺ mV, and a beginning HOCl concentration of 8-10 ppm. The water is preferably sprayed for several seconds at room temperature. This disinfecting step kills harmful microbial agents on the skin due to the antimicrobial capabilities of Type A water. After applying the Type A water, the skin may air dry to complete the process.

### Example 2

Type B water is applied to clean the face, hands or skin, followed by Type C water to disinfect the face, hands, or skin. A fluid wash using Type B water is applied having a pH of 10.5-12.0, an ORP reading of 600⁻ to 950⁻ mV, and a beginning sodium hydroxide concentration in the range of 8-50 ppm. In a preferred embodiment, the Type B water has a pH of 10-11 and an ORP reading of 850⁻ mV. The solution is preferably sprayed for several seconds at room temperature. The spray wash using Type B solution saponifies the lipids on the surface of the skin. Within seconds after saponification from applying the Type B water, the Type C water is applied to the face or hands to disinfect. The Type C water is applied having a pH of 3.5-6.0, an ORP reading of 850⁺ mV to 1150⁺ mV, and a beginning HOCl concentration of 8-70 ppm. In a preferred embodiment, the Type C water has a pH of 4.5-6.0, an ORP reading of 850-1150⁺ mV, and a beginning HOCl concentration of 8-10 ppm. The solution is preferably sprayed for several seconds at room temperature. This disinfecting step kills harmful microbial agents on the skin due to the antimicrobial capabilities of Type C water. After applying the Type C water, the skin may air dry to complete the process.

### Example 3

Type B water is applied to the face, hands, or skin, followed by Type A water, and finally Type C water. A fluid wash using Type B water is applied having a pH of 10.5-12.0, an ORP reading of 600⁻ to 950⁻ mV, and a beginning sodium hydroxide concentration in the range of 8-50 ppm. In a preferred embodiment, the Type B water has a pH of 10-11 and an ORP reading of 850⁻ mV. The solution is preferably sprayed for several seconds at room temperature. The spray wash using Type B water saponifies the lipids on the surface of the skin. Within seconds after saponification from applying the Type B water, the Type A water is applied to the skin to disinfect. The Type A water is applied having a pH of 1.8-3.4, an ORP reading of 1000⁺ mV to 1400⁺ mV, and a beginning HOCl concentration of 8-70 ppm. In a preferred embodiment, the Type A water has a pH of 2.2-2.4, an ORP reading of 1130⁺ mV, and a beginning HOCl concentration of 8-10 ppm. The solution is preferably sprayed for several seconds at room temperature. This disinfecting step kills harmful microbial agents on the skin due to the antimicrobial capabilities of Type A water. Within seconds after applying the Type A water, the Type C water is applied to the skin to disinfect. The Type C water is applied having a pH of 3.5-6.0, an ORP reading of 850⁺ mV to 1150⁺ mV, and a beginning HOCl concentration of 8-70 ppm. In a preferred embodiment, the Type C water has a pH of 4.5-6.0, an ORP reading of 850-1150⁺ mV, and a beginning HOCl concentration of 8-10 ppm. The solution is preferably sprayed for several seconds at room temperature. This disinfecting step kills harmful microbial agents on the skin due to the antimicrobial capabilities of Type C water. After applying the Type C water, the skin may air dry to complete the process.

Alternative methods in addition to those disclosed in detail in Examples 1-3 may feature any combination of Type A, Type B, and Type C water in any series of steps. The result of any such combinations or series of steps will result in the cleaning and disinfecting of the face, hands, or skin without cytotoxicity and skin irritation. Type A, Type B, or Type C water alone, and not in combination with other types of electrolyzed water, also may be applied to the skin. Type B water preferably is applied to the skin when organic matter, inorganic matter, or both are present on the surface of the skin due to the emulsifying and cleaning properties of Type B water. The skin surfaces to which the types of electrolyzed water herein disclosed are applied may include any exterior skin surface of the body. The types of electrolyzed water herein disclosed may be applied to adults, children, and babies, for example, to a baby's bottom. Although spray application is particularly preferred, application of electrolyzed water to the skin also may be accomplished by means of a foam, mist, towel, pad, prepackaged moistened towelette, wipe, or other appropriate means of applying a liquid to the skin. Surfactants may be added to the electrolyzed water, and particularly to Type B water, to create a foam application. Furthermore, disinfecting components such as alcohol, quaternary ammonium (“quats”), or other disinfectants may be added to the electrolyzed water, and particularly to Type B water.

In the specification, there have been disclosed typical preferred embodiments of the invention, and although specific terms are employed, the terms are used in a descriptive sense only and not for purposes of limitation. The invention has been described in considerable detail with specific reference to these embodiments. It will be apparent, however, that various modifications and changes can be made within the spirit and scope of the invention as described in the foregoing specification and as defined in the appended claims.

That claimed is:

1. A method of cleaning and disinfecting skin comprising the step of applying electrolyzed water to the skin.

2. The method of claim 1 wherein the electrolyzed water comprises Type A water.

3. The method of claim 1 wherein the electrolyzed water comprises Type B water.

4. The method of claim 3 wherein the Type B water further comprises surfactants.

5. The method of claim 3 wherein the Type B water further comprises disinfecting components.

6. The method of claim 1 wherein the electrolyzed water comprises Type C water.

7. The method of claim 1 wherein matter is present on the surface of the skin.

8. The method of claim 7 wherein the matter comprises organic matter and the electrolyzed water comprises Type B water.

9. The method of claim 7 wherein the matter comprises inorganic matter and the electrolyzed water comprises Type B water.

10. The method of claim 1 wherein the electrolyzed water is applied to the skin by a means selected from the group consisting of a spray, a foam, a mist, a towel, a pad, a prepackaged moistened towelette, and a wipe.

11. The method of claim 10 wherein the electrolyzed water comprises Type C water.

12. A method of cleaning and disinfecting skin comprising the steps of:
(a) applying Type B water to the skin; and
(b) applying thereafter a type of electrolyzed water not being Type B water to the skin.

13. The method of claim 12 wherein step (b) follows step (a) by seconds.

14. The method of claim 12 further comprising the step of air-drying the skin.

15. The method of claim 12 wherein the electrolyzed water of step (b) comprises Type A water.

16. The method of claim 12 wherein the electrolyzed water of step (b) comprises Type C water.

17. A method of cleaning skin comprising the step of applying Type B water to the skin.

18. The method of claim 17 further comprising the step of applying a type of electrolyzed water not being Type B water to the skin and wherein the skin is cleaned and disinfected.

19. The method of claim 18 wherein the electrolyzed water comprises Type A water.

20. The method of claim 18 wherein the electrolyzed water comprises Type C water.
